(19) United States
(12) Patent Application Publication
(10) Pub. No.: US 2005/0004437 A1
(43) Pub. Date: Jan. 6, 2005

(54) Simulation device for playful evaluation and display of blood sugar levels

(76) Inventors: Heiner Kaufmann, Bern (CH); Thomas Vering, Bremgarten b. Bern (CH)

Correspondence Address: David E. Bruhn, Esq.;DORSEY & WHITNEY LLP, Intellectual Property Department, Suite 1500, 50 South Sixth Street, Minneapolis, MN, 55402-1498, US

(21) Appl. No.: 10/830,857

(22) Filed: Apr. 23, 2004

Related U.S. Application Data
(63) Continuation of application No. PCT/CH02/00573, filed on Oct. 21, 2002.

(30) Foreign Application Priority Data
Oct. 26, 2001 (CH) 1974/01

(51) Int. Cl. A61B 5/00

(52) U.S. Cl. 600/300; 128/905; 345/952

(57) ABSTRACT

A simulation device for playful evaluation and display of blood sugar levels, including a display, wherein the evaluation is displayed by a virtual creature.

### CROSS-REFERENCE TO RELATED APPLICATION(S)

This application is a continuation of International Application No. PCT/CH02/00573, filed on Oct. 21, 2002, which claims priority to Swiss Application No. 1974/01, filed Oct. 26, 2001, the content of both are incorporated in their entirety by reference.

### BACKGROUND

The present invention relates to simulation devices for playful evaluation and display of blood sugar levels.

Diabetics are obliged to check their blood sugar levels several time a day. This is done by sampling blood themselves, applying the blood to a glucose measuring strip, connecting the measuring strip to the measuring and displaying apparatus and lastly displaying the measurement result.

The disadvantages of the known systems are the limited interactivity, and insufficient visual, haptic and acoustic representation of individual measuring results and/or the evaluation of measuring results reached.

### SUMMARY

It is an object of the invention to convey to the patient the information of the individual measuring results and the evaluation of multiple measuring results in order, in a visually, haptically and acoustically appealing—more playful—way

The invention addresses the object using a simulation device featuring a virtual creature which communicates the evaluation to the user in various ways and interacts with the user through signs, speech or other acoustic signals, vibration, changes in temperature, size, shape or colour, and changes in the behaviour of the virtual creature.

The simulation device in accordance with the invention comprises a housing and a display means, for example in the form of a graphic LCD display. Via an input device which is directly attached to the housing or can also be attached to an apparatus separate from the housing, the user inputs his blood sugar levels into a storing, evaluating and controlling unit, for example a microchip or microprocessor, for the purpose of storing the values. These can of course also be separate units. The inputted value is analyzed and assessed by the chip or processor using a stored evaluation method. Depending on the result, the apparatus then communicates the evaluation result and the measured result to the user in a playful way. In some preferred embodiments, the evaluation is displayed with the aid of a virtual creature which conveys the evaluation through signs, speech or other acoustic signals, changes in size, shape or color, or changes in posture or changes in its behavior.

In accordance with another aspect of the invention, which can be used together with or independently of the simulation device described above, the evaluation of the readings is conveyed in the form of heating or cooling the entire apparatus or only a part of it. It is heated or cooled by means of a conventional cooling or heating element.

A number of the parameters mentioned can also display or communicate the evaluation simultaneously or sequentially by being changed.

In order to also be able to operate the apparatus at night, in some preferred embodiments, it comprises a light-emitting diode.

The device in accordance with the invention can also be integrated into a PC, a mobile pocket PC or a mobile telephone

In another embodiment of the device in accordance with the invention, the measured values are transmitted directly from a blood sugar measuring apparatus to the simulation device. The advantage of this arrangement and method is that readings do not have to be read from the measuring system and then inputted into the simulation device via a keyboard. Here, too, the data are transmitted from the measuring system to the simulation device either via a wire or wirelessly.

In another embodiment, the simulation device is integrated into a conventional blood sugar measuring system.

In another embodiment, the device in accordance with the invention exhibits a specific shape and/or choice of material of the housing; if, for example, the device is integrated into a teddy bear, this could increase acceptance of the simulation device by children.

In addition, all the simulation devices can be controlled such that not only individual blood sugar level inputs change the display parameters, but rather by storing multiple values, the longer-term state of health is represented, e.g., the more bent over the virtual creature is displayed, the worse the average value of the readings for the previous month.

Insufficient discipline by the user can also be very easily represented, e.g., the more heavily the virtual creature blushes, the more undisciplined blood sugar levels are transmitted to the simulation device. In this respect, the simulation device can also be described as a monitoring apparatus.

The behavior of the virtual creature can also be changed by means of adjusting parameters. Thus, for example, the number of interactions per day between the virtual creature and the diabetic can be defined.

### BRIEF DESCRIPTION OF THE DRAWINGS

Exemplary preferred embodiments of a device and method in accordance with the invention are described below with the aid of figures, wherein the figures show the following:

FIG. 1 is a schematic representation of a simulation device in accordance with the invention;

FIG. 2a is a schematic representation of the simulation device in accordance with the invention, with an external input keyboard,

FIG. 2b is a schematic representation of the simulation device in accordance with the invention, with an external blood sugar measuring system;

FIG. 3 is a schematic representation of the simulation device in accordance with the invention, integrated into a blood sugar measuring system.

### DETAILED DESCRIPTION OF THE INVENTION

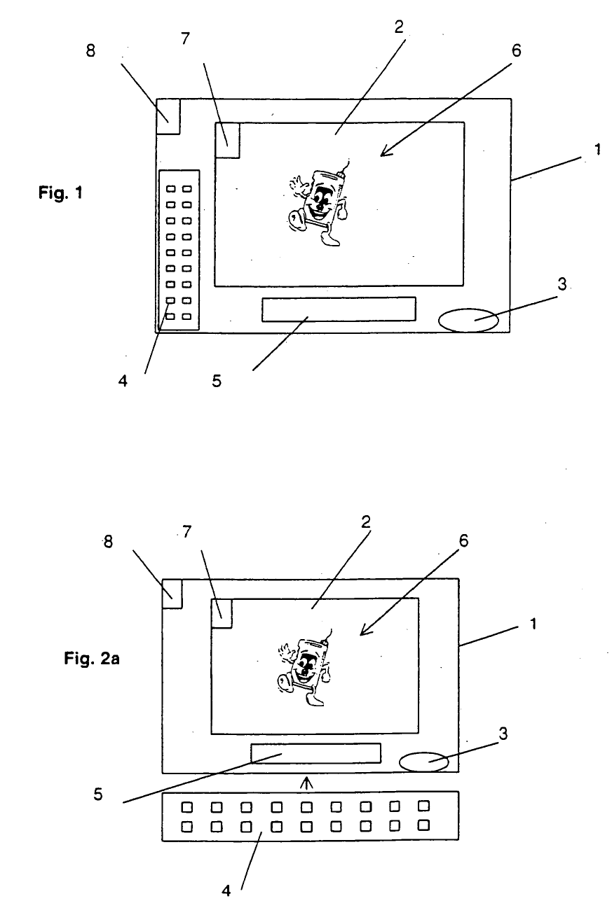

As can be seen from FIG. 1, the simulation device in accordance with the invention comprises a housing 1, a display means 2, for example in the form of a graphic LCD display, and an “on/off” or “reset” button 3. Via the keyboard 4, which can be directly attached to the housing, the user inputs his blood sugar levels into a storing, evaluating and controlling unit 5, for example a suitable microchip or microprocessor, for the purpose of storing the values. The inputted value is then analyzed and assessed by the chip 5 using a stored evaluation method. Depending on the result, the apparatus communicates the evaluation result and the measured result to the user. In some preferred embodiments, the evaluation is displayed with the aid of a virtual creature 6. Acoustic signals are generated by means of a signal emitters 8.

For purposes of and uses in the present invention, exemplary suitable microchips, computers, microprocessors, processing, communication and display systems and electronic devices and components, and methods of their use are described in U.S. Pat. No. 6,186,145, the disclosures and teachings of which patent are incorporated herein by reference in their entirety.

It is also possible to display the evaluation by means of a suitable heating/cooling element (not shown). It is equally possible to display using a suitable vibration element (not shown).

In order to be able to operate the apparatus at night, in some preferred embodiments it comprises a suitable illumination element or elements, such as a light-emitting diode 7.

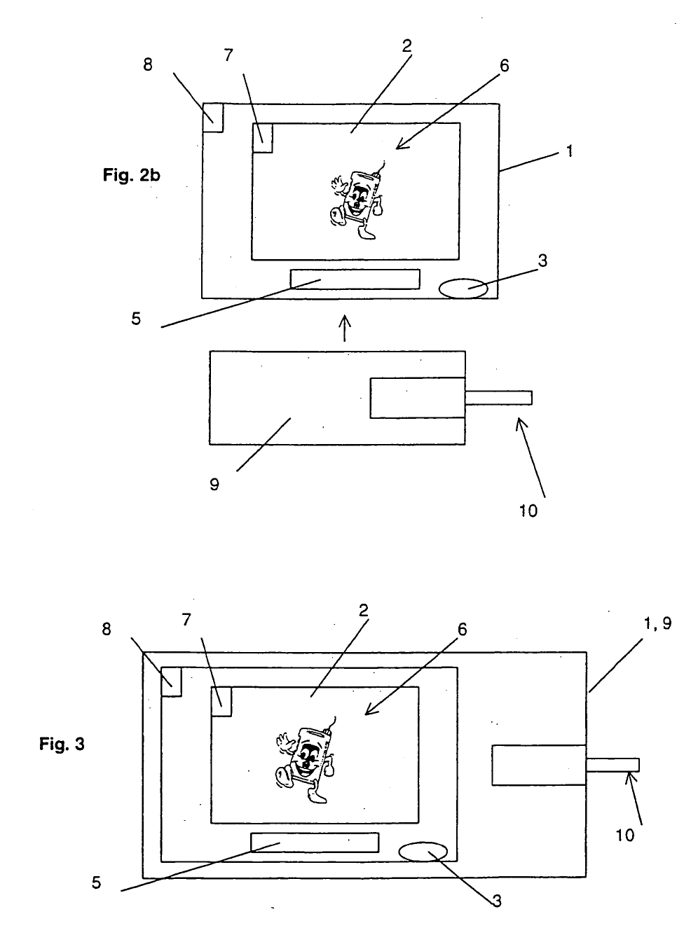

FIG. 2a shows another embodiment of the device in accordance with the invention. The input keyboard is connected to the evaluation and displaying apparatus via wireless communication or by means of wires.

FIG. 2b shows another embodiment of the device in accordance with the invention, in which the measured values are transmitted, wirelessly or by means of wires, from a blood sugar measuring apparatus 9, shown with a measuring strip 10, to the simulation device.

FIG. 3 shows another embodiment, in which the simulation device is integrated into a conventional blood sugar measuring system.

In the foregoing description, embodiments of the present invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A simulation device for displaying and evaluating blood sugar readings, comprising:
a housing;
a display means; and
a storing, evaluating and controlling unit;
wherein the evaluation is displayed by means of a virtual creature.

2. The simulation device as set forth in claim 1, wherein said simulation device has an input device.

3. The simulation device as set forth in claim 1, wherein the evaluation of the blood sugar levels causes a change in said virtual creature.

4. The simulation device as set forth in claim 3, wherein said change in the virtual creature can be seen as a change in color.

5. The simulation device as set forth in claim 1, wherein the device is integrated into another apparatus.

6. The simulation device as set forth in claim 1, wherein the device is integrated into a pocket PC.

7. The simulation device as set forth in claim 1, wherein the device is integrated into a blood sugar measuring apparatus.

8. The simulation device as set forth in claim 1, wherein the evaluation of the blood sugar levels is additionally conveyed via acoustic means.

9. The simulation device as set forth in claim 1, wherein the housing has a specific shape.

10. The simulation device as set forth in claim 1, wherein the ascertained blood sugar levels are transmitted to the evaluating unit by means of wireless communication.
